(19) United States
(12) Patent Application Publication
(10) Pub. No.: US 2008/0176267 A1
(43) Pub. Date: Jul. 24, 2008

(54) Method For Obtaining Primary Culture Tumour Cells From Tumour Tissue, In Particular Breast Carcinoma, Primary-Culture Tumour Cells And Their Use

(76) Inventors: Ralf Hass, Uetze (DE); Hartmut Kueppers, Hannover (DE); Axel Dehn, Langenhagen (DE); Jutta Rogoll, Hannover (DE)

Correspondence Address: WHITHAM, CURTIS & CHRISTOFFERSON & COOK, P.C., 11491 SUNSET HILLS ROAD, SUITE 340, RESTON, VA, 20190, US

(21) Appl. No.: 11/910,541

(22) PCT Filed: Apr. 5, 2006
(86) PCT No.: PCT/DE2006/000608

§ 371 Date: Oct. 3, 2007

(30) Foreign Application Priority Data
Apr. 7, 2005 (DE) 10 2005 015 953.2

(51) Int. Cl. C12Q 1/02; C12N 5/02; C12N 5/00

(52) U.S. Cl. 435/29; 435/378; 435/325

(57) ABSTRACT

The present invention relates to methods for obtaining primary-culture tumour cells from tumour tissue. More precisely, the present invention relates to a method for obtaining primary-culture tumour cells from tumour tissue, in particular breast carcinoma, where, in one method step, the tumour tissue is divided into tumour-tissue sections of a certain size, and these tissue sections are then cultured under defined conditions. The invention also concerns the primary-culture tumour cells that can be obtained by this method from the tumour tissue in particular primary-culture tumour cells from breast carcinoma Finally, the invention relates to the use of these primary-culture tumour cells for, among other things, determining an individual course of tumour treatment, or for testing and screening of new therapeutic agents against tumours.

The present invention relates to methods for obtaining primary culture tumor cells from tumor tissue. More particularly, the present invention relates to a method for obtaining primary culture tumor cells from tumor tissue, in particular breast carcinoma, wherein in one process step the tumor tissue is subdivided into tumor tissue pieces of a defined size and these tissue pieces are then cultured under defined conditions. The invention further concerns the primary culture tumor cells obtainable by this method from the tumor tissue, in particular primary culture tumor cells from breast carcinoma. Finally, the invention relates to the use of these primary culture tumor cells for, inter alia, defining an individual course of tumor therapy or for the testing and screening of new antitumor agents.

### PRIOR ART

Tumors and the corresponding cancer diseases are one of the most frequent causes of death in man. As one form of malignant cancerization of the mammary gland, breast cancer is by far the commonest cancer disease in women. Owing to the only very limited therapeutic options, surgery, chemotherapy/hormone therapy and radiation therapy, with diversified tumor progression, breast cancer is also one of the commonest disease-related causes of death in women. However, other types of cancer, such as bladder cancer, lung cancer, skin cancer and or other types of cancer of epithelial origin and other types of glandular cancer, such as pancreatic cancer, are also a common cause of death in man.

In breast cancer patients, following the operation and the removal of the primary tumor, the breast carcinoma, associated therewith, an adjuvant drug therapy with cytostatic agents is often given. Particularly where metastasization of the primary tumor has already taken place, i.e. for example with simultaneous tumor attack of the axillary lymph nodes, this adjuvant drug therapy is carried out with cytostatic agents. Other therapeutic methods include radiation therapy or hormone therapy or combinations of these forms of therapy. Indeed, hitherto, inter alia the hormone receptor status of the tumor tissue is determined, in order to effect a stratification of the patient, i.e. to determine the strategy for the treatment of the patient. The correct choice of the form of therapy is however very important. Hormone treatments, e.g. with tamoxifen, can only be used for treatment if hormone receptor positive cancer cells, e.g. estradiol-positive breast cancer cells, are detected in the tumor. Moreover, it is well known that a certain percentage of these hormone receptor-positive tumor cells do not respond to the hormone therapy. The same applies for other forms of therapy or even for combined forms of therapy such as simultaneous chemotherapy and hormone therapy or combined chemotherapy and radiation therapy. The correct choice of the form of therapy, the so-called individualized therapy of the diseased person is therefore critical in the treatment of tumor diseases. Particularly as the time since diagnosis of the cancer disease is also an important factor affecting the success of the therapy.

The necessary screening for an adequate form of treatment independently of the surgical intervention, in other words the testing of hormone, chemotherapy and/or radiation therapy measures, was hitherto limited to immortalized breast cancer cell lines (e.g. MCF-7, etc.) in vitro and/or to animal experiments in vivo. However, immortalized cell lines are an artificial test system and do not correspond to the actual in vivo situation. The same applies for animal models, since these are mostly induced tumor animal models. Transfer of the results obtained in the animal model or with immortalized cell lines to the in vivo situation is difficult. The stratification of the patient to be treated therefore still remains a major problem. In particular, multidrug-resistant tumors (MDR tumors) are a major problem in the treatment of a tumor patient.

As already stated above, in the breast cancer field for example the classical prognostic factors in patients with primary breast carcinoma are the tumor size, type of differentiation, hormone receptor status and lymph node attack. In everyday clinical practice, it is seen again and again that tumors which according to these established prognostic factors should by rights be attended by a good prognosis can adopt an aggressive disease course. Thus ca. 30% of patients with a node negative breast carcinoma suffer a relapse within 10 years after the diagnosis. Hence the stratification of the individual patients and thereby the definition of the individual tumor therapy are an essential aspect of the successful treatment of the tumor patient.

As already mentioned above, the in vitro screening methods and the animal experiment methods have only a generalized predictive value, entirely nonspecific with regard to the diversification of the individual case. This has the consequence that broad-brush therapeutic schemes were developed for routine use, which in some patients achieve only inadequate effects or even fail completely. In the worst case, these therapeutic schemes can even induce secondary tumors in healthy proliferating tissue owing to their DNA-damaging and mutagenic action. For this reason, for example in the case of a breast cancer disease, the individual diagnostic aids are extremely limited and moreover there are in principle no options for individualized therapeutic approaches.

As already mentioned above, immortalized cell lines from breast carcinomas are known. As a result of the immortalization, in contrast to in vivo conditions they represent a rather artificial cell culture. Inter alia, these immortalized cell lines differ in that their reaction to active substances, for example for the testing of drugs, is found to be different to the reaction of cells in vivo, i.e. in patients. Thus inter alia, Hass et al. (Signal Transduction, 3, pages 9 to 17, 2003) reported on the differences in the reactions of normal breast epithelial cells (HMEC-1) to elevated estrogen concentration, compared to the breast tumor cell lines MDA-MB-231 and MCF-7.

Attempts have been made to obtain primary culture cells from tumor tissue, e.g. from breast carcinoma. Thus for example Bartsch et al. LifeSciences 67, pages 2953-2960, 2000, describes the study of tumors from patients with mammary and ovarian carcinoma. Here the tumor material was divided into small fragments and to isolate the cells these fragments were digested with enzyme reagents, which usually contain proteases such as trypsin or chemotrypsin and DNases and RNases. These cells isolated by protease digestion thus obtained are then cultured.

However no method is known from the state of the art whereby primary culture tumor cells are simply obtained from the tumor tissue, where these cells have not been altered by immortalization, but can nonetheless be kept in culture for a prolonged period without significant alteration in their properties. That is to say, previously existing methods for the isolation and testing of breast cancer cells also have their limitation in the culturing, e.g. Stampfer M R, J. Tissue Culture Methods, 9: 107-115 (1985).

### SUMMARY OF THE INVENTION

The purpose of the present invention is therefore to provide a method which enables primary culture tumor cells to be obtained from the tumor tissue and thereby overcomes the aforesaid disadvantages, in other words which enables more prolonged culturing of the tumor cells without significant alteration in these. These primary culture tumor cells thus obtained enable the stratification of patients for a therapy but also the use of these obtained cells in other fields, e.g. in the field of screening and testing new potential antitumor agents and in the development and testing of corresponding administration schemes for new and old antitumor agents.

By means of the new method, it has been possible to concentrate and grow over a long period primary culture cells ex vivo from tumor tissue samples from tumors, in particular epithelial tumors such as breast carcinomas.

This direct proliferation of ex vivo tumor primary cultures opens up a range of individual diagnostic and therapeutic possibilities. The present invention thus likewise concerns the use of these tumor primary cultures in screening assays and test systems for the stratification of patients, i.e. for the definition of the individual tumor therapy for the patient. Further, the tumor primary cultures enable the provision of systems for testing and screening new potential antitumor agents and for defining administration schemes for new and old antitumor agents.

Thus in preferred embodiments the process according to the invention comprises the processing of the tumor tissue without an enzymatic digestion, in particular without a protease digestion, before culturing of the tumor tissue. According to the invention, for culturing the tumor tissue is subdivided into tumor tissue pieces of a size with a diameter of about 0.5 mm to about 3 mm. Next, the tumor tissue pieces are cultured in a medium suitable for tumor cells. This medium is preferably supplemented with bovine pituitary extract, hydrocortisone, insulin and human epidermal growth factor. In further preferred embodiments, this medium contains no serum and/or no antibiotics.

Preferably, the tumor tissue is primary breast carcinoma. In embodiments according to the invention, these tumor primary cultures in that case form multilayer cell agglomerations on culturing.

### BRIEF DESCRIPTION OF DIAGRAMS

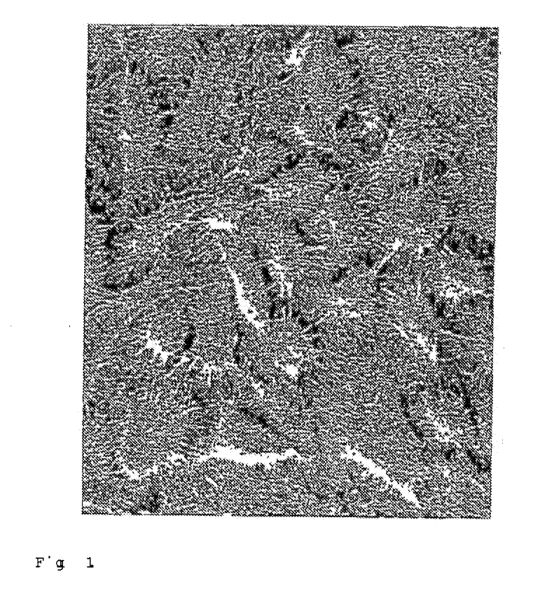

FIG. 1: FIG. 1 is an electron micrograph of breast cancer primary culture cells.

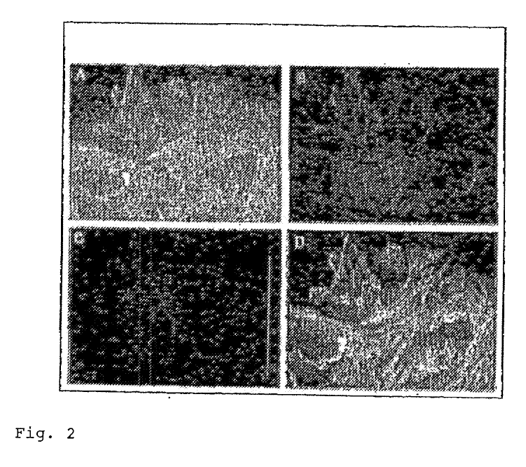

FIG. 2: FIGS. 2A to D are fluorescence pictures of breast cancer primary culture cells which have been labeled with different markers. FIG. 2A: pancytokeratin, FIG. 2B: vimentin, FIG. 2C: DAPI. FIG. 2D shows a superposition of the individual fluorescence pictures shown in FIGS. 2A to C.

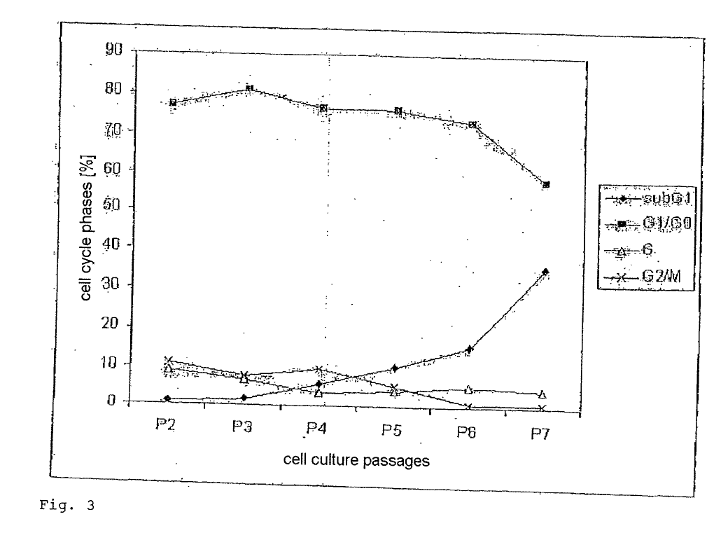

FIG. 3: FIG. 3 is a graph for cell cycle analysis of different culturing passages of breast cancer primary tumor cells. P2 to P7 here mean the respective passages, passage 2 to passage 7.

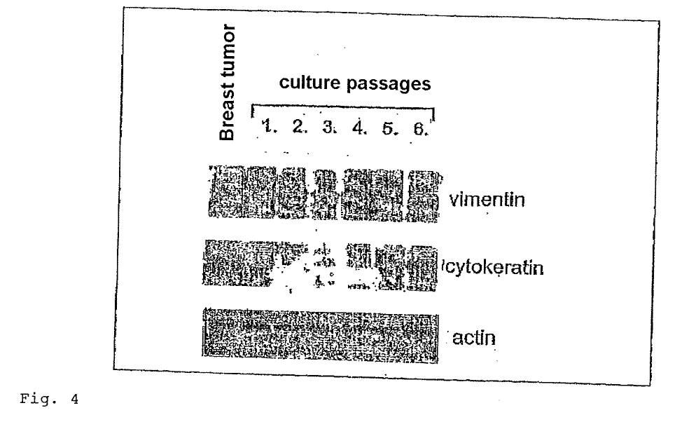

FIG. 4: FIG. 4 is a Western blot in which the expression of pancytokeratin, vimentin and -actin at the protein level in different passages during the culturing of breast cancer tumor cells are shown.

### DETAILED DESCRIPTION OF THE INVENTION

The invention provides a method for obtaining primary culture tumor cells from tumor tissue, in particular tumor primary cultures from breast carcinomas, and primary culture tumor cells obtainable with this method, in particular tumor primary cells of breast carcinoma. The primary culture tumor cells according to the invention, which in a preferred embodiment derive from epithelial cell carcinomas, in particular breast carcinomas, can be obtained from tumor tissue samples by the method according to the invention and multiplied in culture, so that they are available for example as a test system for various applications. At the same time, these primary culture tumor cells obtained can meanwhile be stored in a conserved state.

Below, the term “primary culture tumor cells” is understood to mean cells obtained from tumor tissue which essentially express the same markers and display essentially the same properties as tumor cells in vivo in the tumor tissue from which these primary culture tumor cells derive. Below, the terms “primary culture tumor cells” and “tumor primary cultures” are used synonymously. The primary culture tumor cells are not artificially immortalized (e.g. by viral transformation) cell lines.

The term “tumor” covers all types of tumors, especially solid tumors. In particular, they are solid tumors of epithelial origin, such as breast carcinoma, bladder carcinoma or lung carcinoma and skin cancer and metastases thereof. In other words, the term “tumor” covers not only primary tumors but also tumors formed by metastasization, in particular organ metastases and bone marrow metastases, and cells from relapsing breast cancer tumors. Furthermore, the term “tumor” covers epithelial cell carcinomas of glandular tissue other than the mammary gland, such as for example pancreatic carcinoma.

The expression “antitumor agents” refers to active substances which have an adverse effect on tumor growth. These antitumor agents include both compounds such as chemotherapeutic agents and hormones, and also other agents for the treatment of tumors, such as radiation or non-conservative forms of therapy.

The expression “system” or “test system” as used here concurrently, includes inter alia so-called kits or other units which contain the primary culture tumor cells according to the invention, and directions for performing inter alia the method according to the invention. These systems or test systems can likewise include further components necessary for performing the method according to the invention, such as reagents, vessels, etc. Here the individual components of the kit according to the invention can be packed in separate containers or together in one container. The system according to the invention can for example be a screening assay such as is well known to the person skilled in the art.

The method according to the invention for obtaining tumor primary cultures from tumor tissue such as breast carcinomas starts from tumor tissue which is for example available in a breast cancer operation or when biopsies are taken. This tissue, obtained in as sterile a manner as possible, is if necessary washed under sterile conditions with a wash solution such as PBS or other physiological solutions and then subdivided under sterile conditions into tumor tissue pieces, in order then to culture the tumor tissue pieces in medium suitable for tumor cells.

Here the expression “in medium suitable for tumor cells” means a medium which promotes the growth of the tumor cells in the tumor tissue.

According to the invention, the processing of the resulting tumor tissue for the culturing of the tumor tissue pieces includes no treatment with protease or, in a preferred embodiment, no enzyme treatment whatsoever. In particular, no trypsin treatment of the tumor tissue is performed before culturing of the tumor tissue pieces.

Also, in a preferred embodiment, the medium suitable for the culturing of the tumor cells in the tumor tissue pieces which is be used in the method according to the invention is supplemented with bovine pituitary extract, glucocorticoids such as hydrocortisone, insulin and recombinant human growth factors such as human epidermal growth factor. Particularly preferably, these substances are contained in the medium in amounts of 500 to 5 μg/ml for the bovine pituitary extract, 0.05 to 5 μg/ml for the glucocorticoids such as hydrocortisone, 0.5 to 50 μg/ml insulin and 1 to 100 ng/ml of human epidermal growth factor. Especially preferred is a medium to which have been added 20 to 80 μg/ml, for example 52 μg/ml, of bovine pituitary extract, 0.1 to 2.5 μg/ml, for example 0.5 μg/ml, of hydrocortisone, 1 to 25 μg/ml, for example 5 μg/ml, of insulin and 2 to 50 ng/ml, for example 10 ng/ml, of human epidermal growth factor.

An especially suitable medium is for example the Mammary Epithelial Cell Growth Medium from Promocell GmbH, Heidelberg, Germany, also referred to below as MaCa medium. In a preferred embodiment, 2 ml of bovine pituitary extract (13 mg/ml), 0.5 ml of hydrocortisone (0.5 mg/ml), 0.5 ml of human recombinant epidermal growth factor (10 μg/ml) and 0.5 ml of insulin (5 mg/ml) per 500 ml of culture medium are added to this Mammary Epithelial Cell Growth Medium.

In a further preferred embodiment, this medium suitable for the culturing of tumor cells contains no serum, neither human serum nor fetal calf serum as normally used for cell culturing. In another embodiment, antibiotics are not added to this medium.

Naturally, other media are also suitable, such as for example the MCDB170 medium which is mentioned in Hammond et al., PNAS, 1984, 81: 5435 to 5439.

In a further preferred embodiment, the tumor tissue pieces of the defined size are cultured at a defined density per square centimeter culture vessel area, such as a culture bottle or a culture dish, or per culture volume in medium, preferably in one of the aforesaid media. Here the culturing takes place at 37° C. in an atmosphere containing 5% CO₂ in the culture vessels in suitable appliances such as an incubation cabinet.

Through the subdivision of the tumor tissue to the defined size, it is now possible to obtain primary culture tumor cells. The subdivision of tumor tissue is performed according to the invention such that the tumor tissue pieces have edge lengths in the range from 0.5 mm to 3 mm. In these tumor tissue pieces, the tumor tissue obtained by an OP or from a biopsy is adequately prepared such that on culturing primary culture tumor cells from tumor tissue, in particular tumor tissue deriving from breast carcinoma, are formed.

The subdivision is preferably effected by cutting, for example with a scalpel or scissors, to a size of the tissue pieces wherein these have lengths of one cut edge of at most 3 mm, preferably 2 mm, such as 1.5 mm, more preferably at most 1 mm, where preferably two side edges essentially perpendicular to one another have this length, and still more preferably three cut edges essentially perpendicular to one another have this preferred length. As a result, the defined size into which the tumor tissue is subdivided is a shape wherein the length of at least one cut edge is 0.5 to 2 mm, wherein also the maximal thickness is in the range from 0.5 to 3 mm, preferably 2 to 1 mm. As an example of shapes which these defined sizes of tumor tissue pieces assume, cubes or cuboids with an edge length in the range from 0.5 to 3 mm, preferably 1 to 2 mm, most preferably about 1 mm, can be mentioned.

Preferably, the tissue pieces with the defined dimensions are seeded in a number of 5 to 20 pieces, more preferably 10 to 15 pieces, per 100 cm² area of the culture vessel, sufficient medium being present to cover the tumor tissue pieces sufficiently, preferably to cover them by more than 1 to 2 mm.

In a further preferred embodiment, the tumor tissue pieces subdivided to a defined size are freed of excessively small tissue pieces and single cells such as extraneous cells, e.g. blood cells, or fragments of these individual cells, by filtration, e.g. with a filter with an exclusion limit of 20 μm, more preferably with an exclusion limit of at least 50 μm, e.g. at least 80 μm, in particular at least 100 μm.

Alternatively, the content of fibroblasts in the preparation can be selectively removed before or after subdivision of the tissue or after a first culturing, by known methods such as the MACS anti-fibroblast kit or a comparable method.

The present invention further relates to the isolated primary culture tumor cells obtainable by the method according to the invention. Preferably, these are primary culture tumor cells from a breast carcinoma.

The cells thus obtainable are characterized in that they display many properties of tumor cells in vivo in the tumor tissue. Furthermore, they express many of the marker molecules similarly to those of the tumor cells in vivo, e.g. cytokeratins. More precisely, in a preferred embodiment the primary culture tumor cells express both cytokeratin and also vimentin.

Detailed morphological studies, for example scanning electron micrographs, show that the methods according to the invention described above generate primary cell cultures of breast carcinoma cells which are essentially free from other cell types, in particular free from fibroblasts.

During culturing, under the aforesaid conditions primary cultures grow out of the tumor tissue pieces as adherent cells and on further proliferation form a multilayer, three-dimensional cell culture there. The three-dimensional structure here is already formed during subconfluent growth of the primary culture tumor cells. On attainment of the confluent state in the cell culture, no contact inhibition of proliferation takes place, but rather the cells are not limited in their proliferation. Thereby the primary culture tumor cells according to the invention also differ from for example the normal human mammary gland epithelial cells, which do not form a multilayer, three-dimensional structure, but rather only grow in a monolayer until confluence in the cell culture. Furthermore, it has been shown that these normal cells lose their proliferative capacity on culturing.

Preferably, after the outgrowth of the primary culture tumor cells from the tumor tissue pieces these tissue pieces are removed from the culture vessel.

The conservation of primary culture tumor cells obtained according to the invention can for example be stored in liquid nitrogen by cryoconservation, i.e. slow freezing of trypsinized cells in cryoconservation medium (e.g. 10% DMSO, 10% fetal calf serum in supplemented medium suitable for culturing of tumor cells, as described above).

Following the cryoconservation, primary culture tumor cells according to the invention can again be placed in culture and are ready for tests or further culturing.

Depending on the individual growth of the tumor cells, the primary culture tumor cells multiplied in culture, i.e. cells grown out of the tumor tissue pieces and further multiplied, which attach adhesively to the surface of the culture vessel, can be used for tests in the confluent or subconfluent state, or further multiplied by trypsinization and normal passaging in culture.

In connection with the long-term culturing, and passaging of the primary culture tumor cells according to the invention sometimes necessary during this, it was found that the cells and the cell tissue can grow continually in culture for more than one year without any protease treatment whatsoever, in other words also without trypsinization for the passaging.

During a passaging by trypsinization of the cells and subsequent further culturing, it was found that the properties of the primary culture tumor cells obtained remain essentially unchanged for at most 7 passages, preferably up to 5, more preferably up to 3 passages, i.e. preferably show unchanged or only insignificantly changed properties compared to the cells which are present in vivo in the tumor tissue.

As can be seen from one of the following examples, it has been found that following ca. 7 passages the properties of the cultured primary culture tumor cells, in the case of tumor cells from a breast carcinoma, alter and degenerate to such an extent that the culture begins to die off. This dying off however also confirms that the primary culture tumor cells according to the invention are not artificially immortalized cell lines and that no immortalization of the tumor cells occurs with the method according to the invention.

The primary culture tumor cells thus obtained can be used in test systems for diagnosis, for the stratification of the therapy or for the testing of new and known antitumor agents. In other words, possible uses of these cells according to the invention arise inter alia in active substance research, since compared to the known cell lines the cells according to the invention better represent the in vivo situation and hence also the response in vivo in the tumor tissue. Hence all types of existing and future therapies, such as hormone therapy, cytostatic agents or radiation therapy, can be tested alone or in combination and thus for example an individualized therapy plan can be developed for an individual. Through the simulation of different forms of therapy, different conservative or innovative therapeutic approaches and the possible combination thereof can be tested before use in the patient, and individually adapted.

Furthermore, the cells according to the invention can be used in the testing and screening of new antitumor agents. Thus for example by means of a test system, a so-called bank, which contains at least two different cultures of primary tumor cells from different tumor tissues, for example on an array, the efficacy and also the side-effects of potential new active substances or other physical or biological methods, and the pharmacokinetic efficiencies can be tested on a homogeneous ex vivo material. Preferably, this bank comprises at least ten different, such as at least twenty different cultures of primary tumor cells obtainable by means of the method according to the invention, which each derive from different tumor tissues. Here the tumor tissue can derive from one type of tumor, such as breast carcinoma, or from different types of tumor.

Through proliferation of the individual cultures, sufficient tumor cell material is available for example to compare effects of single and persistent administration of substance and to optimize therapeutic administration time points, i.e. to optimize the administration schemes for the selected tumor therapy.

On account of the aforesaid properties of the primary culture tumor cells obtained, these are also suitable for use in standardized test and screening systems, such as in high throughput screening methods for the development and use of new and known antitumor agents and administration schemes for these in therapeutic measures.

A particular advantage of the method according to the invention is that it is capable of generating tumor primary cultures, for example from breast carcinoma, within a few days and it thus makes it possible rapidly and accurately to draw up an individualized therapy plan for the patient and thus also to dispense with possibly ineffective standard therapies.

### EXAMPLE 1

### Generation of a Primary Cell Culture from Breast Carcinoma

Tumor tissue which was available from a breast cancer operation was washed three times under sterile conditions at room temperature in phosphate-buffered salt solution (PBS) and in MaCA medium (Mammary Epithelial Cell Growth Medium, PromoCell, Heidelberg, Germany) which was supplemented with 2 ml of bovine pituitary extract (13 mg/ml), 0.5 ml of hydrocortisone (0.5 mg/ml), 0.5 ml of recombinant human epidermal growth factor (EGF, 10 μg/ml), 0.5 ml of insulin (5 mg/ml) and 0.5 ml of gentamycin (50 mg/ml), mixed with amphotericin B (50 μg/ml), per 500 ml, cut with a scalpel into tissue pieces with a maximum edge length of 1 to 1.5 mm, which had a cuboid shape.

The tissue pieces, up to 10 to 15 pieces in 50 ml of the aforesaid supplemented MaCA medium, were cultured at 37° C. in a water-saturated 5% CO₂ atmosphere.

After a culturing period of a few days, cell agglomerations which have grown out of the tumor pieces and adhere to the surface of the culture dish, forming multilayer cell agglomerations can be observed.

With conventional trypsin/EDTA solution, the cell agglomerations that have grown on the surface of the culture dish can be detached and subcultured, if necessary after washing and gentle centrifugation. For this, the mixture of the outgrown tumor cells, i.e. the cells growing on the surface of the culture dish mixed with the original tumor tissue is washed twice with PBS at 37° C. and then after treatment with 0.25 percent trypsin/EDTA solution (1:10, 0.25% trypsin/EDTA, Invitrogen GmbH, Karlsruhe, Germany) detached for about 10 to 15 mins at 37° C. The cell suspension is passed through 100 μm filters (DaKoCytomation, Hamburg, Germany) in order to remove the original tumor tissue pieces. The cells contained in the filtrate are described as passage 1 cells, centrifuged down (400×g/5 mins), resuspended in fresh supplemented MaCA medium and cultured in cell culture bottles to a density of ca. 5×10⁴ cells/ml.

The breast cancer primary cells of this first passage can in the subconfluent state be detached by a further trypsin/EDTA treatment, and as passage 2 cells, are then ready as individual breast cancer primary cancer cells in sufficient quantity for function analyses.

### EXAMPLE 2

### Additional Reduction of Fibroblasts in Preparations of Tumor Tissue

In accordance with Example 1, primary cell cultures are generated from tumor tissue, and in addition, the cells of the first passage can be treated with an MACS anti-fibroblast kit (Miltenyi Biotech GmbH, Bergisch Gladbach, Germany).

By means of the monoclonal anti-fibroblast antibodies of the mouse, which are immobilized on colloidally dispersed super-paramagnetic beads, any fibroblasts present can be selectively removed from the primary cell culture of the first passage. Next, the tumor cells are cultured in supplemented MaCA medium according to Example 1.

In the development of this invention, it was however found that an additional removal of fibroblasts from primary cell cultures of the first passage is to a large extent superfluous, since, owing to the method according to the invention, the culture already displays no significant contamination with fibroblasts.

### EXAMPLE 3

### Cryoconservation of Breast Cancer Primary Cell Cultures

Breast cancer primary cell cultures prepared according to Example 1 are trypsinized in supplemented MaCA medium in the normal way after the first, second or third passage, and made up to ca. 5×10⁵ cells/ml in a test-tube with MaCA medium, according to Example 1, and in addition deep frozen to −80° C. with 10% (v/v) fetal calf serum (Invitrogen) and 10% (v/v) DMSO (Sigma) over a period of about 12 hrs and then shock-frozen in liquid nitrogen (−196° C.) and stored.

Shock-frozen primary culture cells can be stored for a short time at −80° C., but preferably in liquid nitrogen. For further culturing or use, the cryoconserved tumor primary culture cells are rapidly warmed to 37° C. and after removal of fetal calf serum and DMSO are cultured in supplemented MaCA medium at 37° C., 5% CO₂.

### EXAMPLE 4

### Characterization of Breast Cancer Primary Cells According to the Invention by Scanning Electron Microscopic Analysis

According to Example 1, an initial cell/tissue culture from tumor tissue pieces from a breast carcinoma, with a maximal edge length in the range from 1 to 2 mm, was cultured on a cover slip and then fixed in a solution containing 3% glutaraldehyde and 2% formaldehyde in 0.1M sodium cacodylate at pH 7.4 for at least 24 hrs at 4° C. Next, the samples were postfixed in 1% OsO₄, then dehydrated in an ethanol gradient. The samples dried at the critical point were coated with gold-palladium (REM coating system E5400, Polaron, Watford, England) and then analyzed at 15 kV in a scanning electron microscope (JEOL SSM-35CF).

A general picture is shown in FIG. 1 at 200 times magnification, in which the culturing of the individual tumor pieces on the glass surface and the overgrowth of the tumor primary cells is visible. The cell morphology of the voluminous cell bodies with strange structures is not typical for contaminating extraneous cells, for example fibroblasts, since the latter have very long-extended and spindle-shaped cell structures.

FIG. 1A also to some extent renders the superimposed layering of the cells within this three-dimensionally growing tumor culture visible, wherein the individual cells form both long and also short cell branches Besides this, it can be seen that the tumor cells form a large number of cytoplasmic branches to their neighboring cells. It is presumed that these inter-cellular links serve for transcellular transport and other intercellular communication processes within the tumor. These intercellular links are also typical for tumor tissue in vivo, so that it can be seen here that the primary cell cultures according to the invention have a structure which very closely resembles the structure of the tumor in vivo.

Overall, the electron micrograph shows that the breast tumor primary culture cells according to the invention form a relatively homogenous population with a three-dimensional structure similar to the cells in the connective tissue agglomeration of a tumor in vivo.

### EXAMPLE 5

### Functional Characterization of Tumor Primary Cultures

Breast tumor tissue is previously known and inter alia apart from the morphology it is characterized by the formation of certain intermediate filaments, in particular the cytokeratins. For the functional characterization of tumor primary cultures according to the invention in MaCA culture medium, cytokeratin was therefore analyzed as one of the markers.

As well as cytokeratins, vimentin intermediate filaments can also be expressed in breast tumor tissue. Expression of vimentin is also typical for fibroblasts, however no cytokeratins whatsoever are found in these.

Thus these two intermediate filaments are both suitable as markers for the identification of possible contamination with other cell types, for example fibroblasts. Fibroblasts are usually a problem in the culturing of primary cells, when fibroblasts display a higher cell division rate than other cell populations and thus overgrow the desired primary cells in the culture.

Specifically, cells of a tumor primary culture were prepared according to Example 1, passaged three times and washed three times for 15 mins on a microscope slide with PBS/Tween-20, then dried for 60 mins. Next the samples were fixed for 10 mins in ice-cold acetone and rehydrated for 5 mins with PBS. Nonspecific binding sites were blocked by incubation in 5% ESA (w/v) in PBS, then incubated for 30 mins with a mouse anti-vimentin antibody (clone V9 (1:100), DakoCytomation). After washing three times with PBS/Tween-20 for 5 mins each time, the incubation with a secondary anti-mouse antibody, labeled with TRITC (1:40, DakoCytomation) is performed. After washing three times with PBS/Tween-20 for 5 mins each time, the samples were treated with mouse serum (1:40, DakoCytomation) for 5 mins and then incubated for 90 mins with an FITC-coupled monoclonal anti-pancytokeratin antibody (clone MNF 116, 1:20, DakoCytomation). After washing three times with PBS/Tween-20 for 5 mins each time, the samples were conserved in DAPI-containing covering medium (Dako-Cytomation) until the immunofluorescence microscopy. As negative controls, parallel cultures were identically treated, except that instead of the mouse anti-vimentin antibody a corresponding mouse serum of the IgG subclass in question was used, which all showed no immunofluorescence.

Fluorescence pictures were all taken with an Olympus SIS F-view II CCD camera in an Olympus IX-50 fluorescence microscope. The analysis of the fluorescence pictures and the superposition of the fluorescence pictures were effected using the image processing program SIS bundle analySISB (Olympus). In the analysis, particularly at the individual cell level, it could be shown that the expression of cytokeratins and vimentin is not distributed in two different cell types, but that detected vimentin is always expressed in an individual cell together with cytokeratins, so that in every case the combination of these two intermediate filament types is an exclusion criterion for fibroblasts. For this, a triple fluorescence dye method was used, the respective individual fluorescence pictures, shown in FIGS. 2A-C, and the corresponding superposition, shown in FIG. 2D, being used for the analysis.

In FIG. 2A, the picture of the green fluorescence of the cytokeratin filaments in the tumor primary culture is shown, in FIG. 2B in red fluorescence, the vimentin filaments and in FIG. 2C the cell nuclei of the respective tumor primary culture cells in blue DAPI staining. The superposition of the individual pictures of FIGS. 2A-C produced with the image processing program bundle analySIS'B (Olympus) is shown in FIG. 2D. FIG. 2D makes it clear that since tumor primary culture cells either express only cytokeratin or display more or less strong co-expression of the vimentin and cytokeratin filaments, the intracellular location of which is however different. These pictures are a significant confirmation that, irrespective of the morphology of the cells illustrated, no fibroblasts are detectable in the culture according to the invention, since all cells at least express cytokeratins.

### EXAMPLE 6

### Use of Breast Tumor Primary Culture Cells for the Analysis of the Efficacy of Drugs against Tumor Tissue in vivo

Breast cancer primary culture cells according to Example 1 or cryoconserved cancer primary cell cultures according to Example 3 were characterized in more detail precisely like the original tumor tissue on the basis of further markers, for example Ki-67, and it was found that tumor primary culture cells according to the invention display a very similar expression pattern of further markers to the tumor tissue isolated from the patient. The cryoconservation of the cell cultures did not alter this property.

For efficacy tests of substances which it might be possible to use as drugs, tumor primary culture cells according to the invention of the second to fourth passage are preferably used. For it has been found that the primary cell cultures in later passages contain a higher proportion of cell cycle-arrested and apoptotic cells, which clearly implies that the cell properties are deviating from the original ones.

Individual tumor primary culture cells were exposed to various chemotherapeutic agents in different concentrations and the efficacy of the compound in question could be estimated on the basis of the alteration in their morphology or apoptosis. On the basis of the great similarity of the tumor primary culture cells to the properties of the tumor tissue in vivo, the result of this ex vivo test can be directly included with the expected therapeutic result in the patient.

Since the method according to the invention is capable of generating tumor primary cultures, for example from breast carcinoma, within a few days, it is suitable for providing a test system for individual female patients, with which the effect of drugs, for example of chemotherapeutic agents, and also the possible result of radiation therapy, can be tested. In this manner, tumor primary culture cells, using which the effectiveness of a large number of drugs or other therapeutic methods can be tested, can be generated from a tumor biopsy. On the basis of the response of the primary cell cultures ex vivo, the therapeutic approach against the original tumor can be assessed individually, so that effective forms of therapy can be selected, while ineffective standard therapies can be dispensed with.

### EXAMPLE 7

### Characterization of the Tumor Primary Culture Cells by Analysis of the Cell Cycles in Successive Culture Passages

For the characterization of the passaged tumor primary culture cells, their cycle was determined by determination of their DNA content in FACS measurements.

In FIG. 3, the results of the measurements are shown in graph form. The generation of the tumor primary culture cells was performed according to Example 1, and the passaging was effected by trypsinization, washing three times in PBS and inoculation at 10⁵ cells/ml in supplemented MaCA medium.

The data shown in FIG. 3 make it clear that the cell properties degrade following the 5^{th} phase. This is shown by the decline in the number of cells which are in the S phase of the cell cycle and the decline in the ability to divide in the course of the successive passages. Beyond the 5^{th} to 7^{th} passage, the G2/M phase is no longer detectable, while at the same time a continuous rise in the population in the sub-G1 phase, which mainly indicates apoptotic cells, is detectable.

### EXAMPLE 8

### Characterization of the Tumor Primary Culture Cells in the Course of Passaging by Western Blotting

Cell samples which derive from passages in Example 6 were analyzed for pancytokeratin, vimentin and -actin by Western blotting.

For the analysis, cells obtainable from the individual passages in Example 6 were washed in PBS, centrifuged down, homogenized in SDS loading buffer with a cannula and then separated by electrophoresis in a denaturing polyacrylamide gel. The proteins were transferred onto ImmobilonP membranes and after blocking with BSA were detected with specific antibodies against pancyto-keratin, and vimentin, and against -actin as loading control. The result is shown in FIG. 4 for pancyto-keratin, vimentin and β-actin. Equal quantities of protein (10 μg) were applied in each case; the fragment sizes stated were determined by means of marker proteins separated in parallel.

The detection of the pancytokeratin shows that cyto-keratin is first expressed in significantly higher amount with increasing culture passage number, in other words, when the primary culture has already reached a growth-arrested state. The expression pattern of pancyto-keratin shows that cytokeratin positive cells mainly have non-malignant properties, so that the expression of cytokeratins in tumor tissue clearly is not a marker for identifying still malignant metastatic tumor cells or tumor relapses.

The detection of the β-actin as control shows that approximately equal quantities of protein were applied.

The expression of vimentin confirms the results from the immunofluorescence (FIG. 2), that together with cytokeratins a co-expression of vimentin is sometimes detectable in the breast cancer primary cultures, whose expression pattern does not however alter significantly in the course of the culture passages.

Further, it could be shown that treatment of the primary culture tumor cells obtained according to the invention with known antitumor agents induced the death of the cells. Thus the primary culture tumor cells showed the expected response, which is further indication that these are primary cultures of tumor cells.

1. A method for obtaining essentially primary culture tumor cells from tumor tissue comprising the steps
a) subdivision of the tumor tissue into tumor tissue pieces of a size with a diameter of about 0.5 mm to about 3 mm and
b) culturing the tumor tissue pieces in medium suitable for tumor cells, characterized in that up to the culturing of the tissue pieces the tumor tissue or the tumor tissue pieces is/are subjected to no treatment with proteases.

2. The method as claimed in claim 1, characterized in that no trypsin treatment is performed before culturing of the tumor tissue pieces in medium suitable for tumor cells.

3. The method as claimed in claim 1, characterized in that no enzymatic treatment of the tumor tissue or of the tumor tissue pieces is performed before culturing of the tumor tissue pieces in medium suitable for tumor cells.

4. The method as claimed in claim 1, characterized in that before the subdivision of the tumor tissue a sterile washing of the tumor tissue is performed.

5. The method as claimed in claim 1, characterized in that the tumor tissue pieces are cultured in a number of 5 to 20 per 100 cm² area of a culture vessel.

6. The method as claimed in claim 1, characterized in that before the step of culturing the tumor tissue pieces smaller fragments or individual cells are removed by filtration with a pore size of 20 to 100 μm.

7. The method as claimed in claim 1, characterized in that the medium for culturing the tumor tissue pieces is not supplemented with serum.

8. The method as claimed in claim 1, characterized in that the medium for culturing the tumor tissue pieces is not supplemented with antibiotics.

9. The method as claimed in claim 1, characterized in that the medium is supplemented at least with bovine pituitary extract, hydrocortisone, insulin and/or human epidermal growth factor.

10. The method as claimed in claim 1, characterized in that the tumor tissue is an epithelial cell carcinoma.

11. The method as claimed in claim 1, characterized in that the tumor tissue is a breast carcinoma, lung carcinoma or bladder carcinoma.

12. The method as claimed in claim 1, wherein the culture medium is a medium suitable for breast carcinoma.

13. The method as claimed in claim 1, characterized in that after an outgrowth of primary culture tumor cells from the tissue pieces the tumor tissue pieces are removed from the culture vessel.

14. The method as claimed in claim 1, characterized in that the primary culture tumor cells form multilayer cell agglomerations on culturing.

15. The isolated primary culture tumor cells obtainable by a method as claimed in claim 1.

16. The primary culture tumor cells as claimed in claim 15, which are primary culture tumor cells from a breast carcinoma.

17. The primary culture tumor cells as claimed in claim 15, characterized in that the cells co-express either only cytokeratins or vimentin and cytokeratin.

18. The primary culture tumor cells as claimed in claim 15 characterized in that they form a multilayer three-dimensional structure even in the subconfluent state.

19. The use of primary culture tumor cells obtainable as claimed in claim 1 for the testing of compounds as antitumor agents against tumor types from which these primary culture tumor cells derive.

20. The use of primary culture tumor cells of an individual obtainable as claimed in claim 1 for the definition of the individual tumor therapy of this individual.

21. The use of the primary culture tumor cells obtainable as claimed in claim 1 in a method for the determination of the metastasization potential of these cells.

22. The use of the primary culture tumor cells obtainable as claimed in claim 1 for the development and testing of administration schemes for antitumor agents.

23. The use of at least two types of primary culture tumor cells from at least two different tumors obtainable as claimed in claim 1 in a test system for testing potential antitumor agents.

24. A test system for testing and/or screening new potential antitumor agents containing primary culture tumor cells obtainable as claimed in claim 1.

25. A system for definition of the individual tumor therapy of an individual containing primary culture tumor cells of this individual obtainable as claimed in claim 1.

26. The system as claimed in claim 24, characterized in that this system contains primary culture tumor cells from at least two different tumors.

27. The system as claimed in claim 24 further containing cell culture medium for the culturing of the primary culture tumor cells.
